(19) United States
(12) Patent Application Publication
(10) Pub. No.: US 2014/0037685 A1
(43) Pub. Date: Feb. 6, 2014

(54) ADJUVANTS THAT ACTIVATE ADAPTIVE IMMUNE SYSTEM BY STIMULATING NLRP3

(71) Applicant: Albert Einstein College of Medicine of Yeshiva University, Bronx, NY (US)

(72) Inventors: Jurgen Brojatsch, Brooklyn, NY (US); Lee Jacobson, Bronx, NY (US)

(73) Assignee: Albert Einstein College of Medicine of Yeshiva University, Bronx, NY (US)

(21) Appl. No.: 13/956,780

(22) Filed: Aug. 1, 2013

Related U.S. Application Data
(60) Provisional application No. 61/723,514, filed on Nov. 7, 2012.
(60) Provisional application No. 61/679,936, filed on Aug. 6, 2012.

(51) Int. Cl. A61K 39/02; A61K 31/35; A61K 31/7076; G01N 33/50; G01N 33/68

(52) U.S. Cl. CPC A61K 39/02; G01N 33/5014; G01N 33/6869; A61K 31/7076; A61K 31/35
USPC 424/234.1; 435/40.5; 435/7.92

(57) ABSTRACT

A method of identifying an agent, or combination of agents, as a candidate immunological adjuvant is provided comprising contacting a cell comprising a Nod-like receptor (Nlrp3) with the agent. Methods of enhancing immune responses to vaccines are also provided.

### CROSS-REFERENCE TO RELATED APPLICATIONS

This application claims benefit of U.S. Provisional Application No. 61/732,514, filed Nov. 7, 2012, and claims benefit of U.S. Provisional Application No. 61/679,936, filed Aug. 6, 2012, the contents of each of which are hereby incorporated by reference.

### STATEMENT OF GOVERNMENT SUPPORT

This invention was made with government support under grant number 1R56A1092497-01A1 awarded by the National Institutes of Health. The government has certain rights in the invention.

### BACKGROUND OF THE INVENTION

Throughout this application various publications, patents, patent application publications and books are referred to, including by number in parentheses. Full citations for the publications may be found at the end of the specification. The disclosures of the publications, patents, patent application publications and books are hereby incorporated by reference in their entirety into the subject application to more fully describe the art to which the subject invention pertains.

The Nod-like receptor (NLR) Nlrp3 is an intracellular surveillance receptor that is critical for the host immune response. Activation of Nlrp3 is essential for the development of a protective immune response against multiple microbial pathogens (1-3). Upon activation, Nlrp3 triggers the sequential recruitment of downstream proteins, such as the adaptor protein Asc and the cysteine protease caspase-1, and the formation of a high-molecular inflammasome complex (4). Generation of this complex results in the auto-catalytic activation of pro-caspase-1 (5). Activated caspase-1 has been shown to trigger two proinflammatory processes: caspase-1-mediated cell death (pyroptosis), and processing of the proinflammatory cytokines IL-1β, IL-18 and IL-33 (6). Intriguingly, caspase-1-associated cytokines have been implicated in adaptive immune responses (7-10). While alum has been shown to activate Nlrp3, and trigger a Nlrp3-dependent immune response, recent studies with alum have challenged the role of Nlrp3 in alum's adjuvant activities (11, 12).

While most pattern recognition receptors respond to a relatively narrow subset of ligands, Nlrp3 has been shown to activate in response to a wide range of bacterial and viral pathogens (13-22). Nlrp3 has also been activated by a range of noninfectious agents, such as pathogen-associated molecular patterns (PAMPs), insoluble particles, and a number of immunologic adjuvants (11, 23-35). It is unclear how these structurally and chemically diverse inducers activate the Nlrp3 inflammasome. It is generally assumed that these compounds do not interact directly with Nlrp3, consistent with a lack of detectable interactions between these agents and the receptor. It is therefore believed that these inducers act indirectly, and trigger one or more common upstream events critical for Nlrp3 signaling (36). Several events have been suggested, including mitochondrial and lysosome disruption (37).

Of these models, lysosome rupture has been frequently implicated as an upstream signal for Nlrp3 activation. Insoluble particulate compounds such as silica, monosodium urate, calcium pyrophosphate dehydrate, and alum, the predominant adjuvant used in the US, have been shown to induce lysosome rupture and to activate Nlrp3 (33). Following endocytosis, accumulation of particulates in phagolysosomes has been shown to destabilize lysosomal integrity. The ensuing release of the lysosomal proteins, including cysteine cathepsins, into the cytoplasm has been suggested to trigger Nlrp3 inflammasome activation (38). Accordingly, cathepsin inhibitors have been shown to block Nlrp3 signaling and caspase-1 activation by several Nlrp3 agents (33, 38). In addition, lysosome rupture triggered by a lysosome-destabilizing dipeptide and by hypertonic solutions has also been shown to trigger caspase-1 activation (33). However it remains unclear the extent to which lysosome rupture contributes to Nlrp3 activation by two commonly studied activators: the potassium efflux inducers, ATP and nigericin.

The present invention addresses the need for novel and improved adjuvants based on Nlrp3 stimulation.

### SUMMARY OF THE INVENTION

A method is provided of identifying an agent, or combination of agents, as a candidate immunological adjuvant comprising contacting a cell comprising a Nod-like receptor (Nlrp3) with the agent, combination of agents, quantifying the Nlrp3 response, comparing the Nlrp3 response to a predetermined level, and determining if the agent, or combination of agents, is a candidate immunological adjuvant, wherein the agent, or combination of agents, is a candidate immunological adjuvant if it effects a Nlrp3 response above a predetermined level of Nlrp3 response, and is not identified as a candidate immunological adjuvant if it effects a Nlrp3 response below the predetermined level of Nlrp3 response or if it does not effect a Nlrp3 response.

A method is also provided of identifying an agent, or combination of agents, as an immunological adjuvant comprising administering to a subject an agent, or combination of agents, identified as a candidate immunological adjuvant by the method above and quantifying a subsequent Th1 response in the subject, and identifying the agent, or combination of agents, as an immunological adjuvant, wherein the agent, or combination of agents, is an immunological adjuvant if it effects a Th1 response in the subject above a predetermined level of Th1 response, and is not identified as an immunological adjuvant if it effects a Th1 response in the subject below the predetermined level of Th1 response or does not effect a Th1 response.

A method of improving the efficacy of a vaccine comprising administering to a subject who is receiving, has received or will receive the vaccine, an amount of a secondary inducer of Nlrp3 effective to improve the efficacy of the vaccine.

Additional objects of the invention will be apparent from the description which follows.

### BRIEF DESCRIPTION OF THE DRAWINGS

FIG. 1A-1B. Alum and LLOMe are poor inducers of inflammasome-associated cytokines. C57BL/6 macrophages were primed with 250 ng/ml LPS to induce IL-1β production for 2 hours. Macrophages were then exposed to increasing concentrations of alum, LLOMe, ATP, or nigericin for 4 hours, or alum for 6 hours, in the presence or absence of the 40 μM Boc-D-CMK or 100 μM CA-074-Me. IL-1β release was assessed by ELISA (A), and cell death was determined by CytotoxOne LDH activity assay (B) from supernatants. All PI measurements were taken in triplicate from representative experiment. FIG. 2. Alum and LLOMe cause depletion of caspase-1 and NLR-associated cytokines. BALB/C macrophages challenged with 5 mM ATP, 500 ng/ml LT, 2.5 mM LLOMe, or 150 ng/ml alum for various times in the presence or absence of 100 μM CA-074-Me. Lysates were probed for pro-caspase-1, IL-18, and IL-1β, and supernatants were probed for IL-1β by immunoblotting. Data is representative of 3 experiments.

FIG. 3A-3D. LLOMe-mediated inflammasome depletion is independent of Nlrp3 signaling. (A) Wild type macrophages and Nalp3/Asc-deficient macrophages were challenged with LLOMe (2.5 mM) in the absence and presence of LPS, the caspase-1 inhibitor Boc-d-cmk or the cathepsin B inhibitor CA-074-Me. Caspase-1 and actin levels were determined 2 hours post LLOMe exposure. (B-C) Wild-type, and Nalp3/Asc/caspase-1-deficient C57BL/6 macrophages were primed with 250 ng/ml LPS to induce IL-1β and Nalp3 for 2 hours. Cells were then challenged with 2.5 mM LLOMe (B) or 10 mM nigericin (C) for 2 hours in the presence or absence of 100 μM CA-074-Me, and cell death was measured using PI exchange assays (top row). Lysates and supernatants were examined for actin and IL-1β levels by Western blot (bottom row). (D) Wild type and Nalp3, Ipaf and Asc-deficient C57BL/6 macrophages were primed for 2 hours with 250 ng/ml of LPS and challenged with 150 ng/ml alum for 8 hours or 2.5 mM LLOMe for 4 hours in the presence or absence of 100 μM CA-074-Me. Membrane impairment was detected by propidium iodide exclusion assay. Nalp3, Ipaf and Asc-deficient macrophages were treated with varying doses of ATP, and nigericin, for 3 hours in the presence or absence of 100 μM CA-074-Me. Plasma membrane impairment was determined by propidium iodide exclusion assays.

FIG. 4A-4C. Specific cathepsins control alum and LLOMe-mediated inflammasome depletion. (A-B) C57BL/6 macrophages were with 250 ng/ml LPS and with increasing amounts of CA-074-Me for 2 hours, and were then challenged with 2.5 mM LLOMe (A) or 10 mM nigericin (B). Cell death was measured by propidium iodide exclusion assay, and protein profiles were determined by immunoblotting from lysates and supernatants. Representative experiment is shown, and PI measurements were performed in triplicates. (C) C57BL/6 macrophages from different cathepsin knockout mice were primed with 250 ng/ml LPS and with increasing concentrations of LLOMe for 2 hours. Cell death was measured by propidium iodide exclusion assay, and IL-1β profiles were determined by immunoblotting from lysates. Representative experiment is shown, and PI measurements were performed in triplicates.

FIG. 5A-5C. Lysosome rupture precedes cell death by alum and LLOMe, but not pyroptosis inducers. (A) Confocal microscopy of lysosome rupture in macrophages treated with 150 ng/ml alum, 2.5 mM LLOMe, or 15 μM nigericin in the absence or presence of 100 μM CA-074-Me. The cells were stained with Hoechst (blue), 20 MW FITC-Dextran (green), CellMask Orange (red), TO-PRO-3 (white). 2D-fluorescent intensity plots of FITC-dextran staining of representative cells derived from alum, LLOMe, and nigericin-treated cells. Flow cytometry analysis of LLOMe and LT-treated cells. BALB/c-derived macrophages were exposed to 2.5 mM LLOMe (B) or 500 ng/ml LT (C) and lysosome and membrane integrity were measured using LysoTracker and PI at different time points using flow cytometry.

FIG. 6A-6D. LPS triggers strong production of Th1 and Th2-associated IgG subtypes. IgG1 (A) and IgG2c (B) production in C57BL/6 mice 3 weeks after subcutaneous challenge with increasing amounts of LPS. (C) A combination of LPS and ATP triggers a strong IL-1β response in vitro and in vivo. Primary C57BL/6 macrophages were treated with 250 ng/ml LPS or PBS for 2 hours, followed by to 5 mM ATP in the presence or absence of the 40 μM Boc-D-CMK. IL-1β release was assessed by ELISA from supernatants. (D) C57BL/6 mice were i.p. primed with 1 ug LPS or PBS for 2 hours, followed by i.p. challenge with 100 mM ATP or PBS. IL-1β production in the intraperitoneal lavage was determined by ELISA 30 min after ATP/PBS challenge.

FIG. 7. Alum and LLOMe mediate inflammasome-independent cell death. Wild type and Nalp3, Ipaf, and Asc-deficient C57BL/6 macrophages were primed for 2 hours with 250 ng/ml of LPS and challenged with 150 ng/ml alum for 8 hours or 2.5 mM LLOMe for 4 hours in the presence or absence of 100 μM CA-074-Me. Membrane impairment was detected by propidium iodide exclusion assay. Nalp3, Ipaf, and Asc-deficient macrophages were treated with varying doses of ATP, and nigericin, for 3 hours in the presence or absence of 100 μM CA-074-Me. Plasma membrane impairment was determined by propidium iodide exclusion assays.

FIG. 8. C57BL/6-derived macrophages were exposed to LPS, and challenged with 2.5 mM LLOMe or 20 mM nigericin in the absence and presence of CA-074-Me. Caspase-1 and actin levels were determined at different time points post LLOMe/nigericin exposure.

FIG. 9. Wild-type macrophages and Nalp3/Asc-deficient macrophages were exposed to LPS, and challenged with alum in the absence and presence of the cathepsin B inhibitor CA-074-Me. Levels of IL-1β and actin were determined at different time points post alum exposure.

FIG. 10. 2D-DIGE gel of proteins isolated from untreated, nigericin- and LLOMe-treated macrophages. C57BL/6 macrophages were primed with 250 ng/ml for 2 hours, and then challenged with 2.5 mM LLOMe or 10 mM nigericin for 90 minutes. Protein lysates from control, nigericin, and LLOMe treated cells were isolated, and subsequently labeled with Cy2, Cy3 and Cy5, respectively, and separated on a 2D-DIGE gel.

FIG. 11. Similar in vitro and in vivo profiles of LLOMe and LPS/ATP-treated macrophages. C57BL/6 mice were i.p. primed with 1 μg LPS or PBS for 2 hours, followed by i.p. challenge with 500 μl of PBS containing 100 mM ATP, 2 mM LLOMe, or PBS only. IL-1β production and LDH activity was determined from the intraperitoneal lavage by ELISA 30 min after challenge.

### DETAILED DESCRIPTION OF THE INVENTION

A method is provided of identifying an agent, or combination of agents, as a candidate immunological adjuvant comprising contacting a cell comprising a Nod-like receptor (Nlrp3) with the agent, combination of agents, quantifying the Nlrp3 response, comparing the Nlrp3 response to a predetermined level, and determining if the agent, or combination of agents, is a candidate immunological adjuvant, wherein the agent, or combination of agents, is a candidate immunological adjuvant if it effects a Nlrp3 response above a predetermined level of Nlrp3 response, and is not identified as a candidate immunological adjuvant if it effects a Nlrp3 response below the predetermined level of Nlrp3 response or if it does not effect a Nlrp3 response.

In an embodiment, the method is performed in vitro. In an embodiment of the methods, the agent is a molecule of 2000 daltons or less. In an embodiment of the methods, the molecule is inorganic. In an embodiment of the methods, the molecule is organic. In an embodiment, the agent is not a polymer or an oligomer. In an embodiment, the agent is a polymer or an oligomer.

A method is also provided of identifying an agent, or combination of agents, as an immunological adjuvant comprising administering to a subject an agent, or combination of agents, identified as a candidate immunological adjuvant by the method therefor described hereinabove or below and quantifying a subsequent Th1 response in the subject, and identifying the agent, or combination of agents, as an immunological adjuvant, wherein the agent, or combination of agents, is an immunological adjuvant if it effects a Th1 response in the subject above a predetermined level of Th1 response, and is not identified as an immunological adjuvant if it effects a Th1 response in the subject below the predetermined level of Th1 response or does not effect a Th1 response in the subject.

In an embodiment, the Nlrp3 response is pyroptosis. In an embodiment, the Nlrp3 response is caspase-1-dependent pyroptosis. In an embodiment, the Nlrp3 response is caspase-1-dependent necrotic cell death. In an embodiment, determining whether the pyroptosis/cell death is caspase-1-dependent is determined by contacting the cells being quantitated for an Nlrp3 response with a caspase-1 inhibitor. The reduction or prevention of the pyroptosis/necrotic cell death by the caspase-1 inhibitor indicates that the cell death is caspase-1-dependent. In an embodiment, the cell is a macrophage. In an embodiment, the cell is a macrophage genetically manipulated to lack Nlrp3 or a Nlrp3 component. In a further embodiment, the cell is a human macrophage.

In an embodiment, the methods further comprise contacting a T-cell with the agent, or combination of agents, and determining T-cell proliferation, wherein an agent or combination of agents which effects T-cell proliferation is a candidate immunological adjuvant.

In an embodiment, the methods further comprise administering to the subject a vaccine or an antigen with the agent or with the combination of agents. In an embodiment, the antigen is a component of a pathogen. In a further embodiment, the pathogen is a pathogen of a mammal. In a further embodiment, the pathogen is a pathogen of a human. In a further embodiment, the pathogen is a virus or a bacterium.

In an embodiment, the methods further comprise determining antibody production subsequent to the administering of agent, or combination of agents.

In an embodiment, the cell is a macrophage. In an embodiment, the cell is a human macrophage.

In an embodiment of the methods, the combination of agents are used. In an embodiment of the methods, a single agent is used.

In an embodiment of the methods, the combination of agents comprises at least one of a potassium efflux inducer, ATP, Bz-ATP, or nigericin.

A method is also provided of improving the efficacy of a vaccine comprising administering to a subject who is receiving, has received or will receive the vaccine, a secondary inducer of Nlrp3. In an embodiment, the subject administered the vaccine and the secondary inducer of Nlrp3 is not administered an additional immune modulator. The subject administered the vaccine and the secondary inducer of Nlrp3 is not administered an additional immune modulator which is an adjuvant. In one embodiment, administering an immune modulator (in addition to the vaccine and secondary inducer of Nlrp3) would be considered as materially affecting the basic and novel properties of the invention.

In an embodiment, improving efficacy comprises increasing one or more immune response parameters for given dose of vaccine as compared to said immune response parameter(s) without the secondary inducer. In an embodiment, improving efficacy comprises effecting a reduction in the amount of vaccine required to achieve one or more immune response parameters as compared to said immune response parameter(s) without the secondary inducer.

In an embodiment, the secondary inducer of Nlrp3 is ATP, Bz-ATP, or nigericin. In an embodiment, the secondary inducer of Nlrp3 is administered in a composition which also comprises the vaccine.

In an embodiment, the secondary inducer of Nlrp3 is an agent or combination of agents identified as a candidate immunological adjuvant by one or more of the methods therefor described herein.

In an embodiment, the vaccine is a lipopolysaccharide (LPS) vaccine. In an embodiment, the vaccine is a gram-negative bacteria lipopolysaccharide (LPS) vaccine. In an embodiment, the vaccine is a vaccine for anthrax (e.g. AVA (BioThrax); chickenpox (Varicella) (e.g. VAR (Varivax); MMRV (ProQuad)); MMR and MMRV; diphtheria (e.g. TaP (Daptacel, Infanrix), Td (Decavac, generic), DT (generic), Tdap (Boostrix, Adacel), DTaP-IPV (Kinrix), DTaP-HepB-IPV, Pediarix), DTaP-IPV/Hib (Pentacel), DTaP/Hib); hepatitis A (e.g. HepA (Havrix, Vaqta), HepA-HepB (Twinrix)); hepatitis B (e.g. HepB (Engerix-B, Recombivax HB), Hib-HepB (Comvax), DTaP-HepB-IPV (Pediarix), HepA-HepB (Twinrix)); HIB (e.g. Hib (ActHIB, PedvaxHlB, Hiberix), Hib-HepB (Comvax), DTaP/Hib, DTaP-IPV/Hib (Pentacel)); HPV (e.g. HPV4 (Gardasil), HPV2 (Cervarix)); influenza (e.g. TIV (Afluria, Agriflu, FluLaval, Fluarix, Fluvirin, Fluzone, Fluzone High-Dose, Fluzone Intradermal), LAIV (FluMist)); Japanese encephalitis (e.g. JE (Ixiaro)); Lyme disease; measles (e.g. MMR (M-M-R II), MMRV (ProQuad)); meningococcal (e.g. MCV4 (Menactra), MPSV4 (Menomune), MODC (Menveo)); Mumps (e.g.MMR (M-M-R II), MMRV (ProQuad)); pertussis (e.g. DTaP (Daptacel, Infanrix);Tdap (Adacel, Boostrix); DTaP-IPV (Kinrix); DTaP-HepB-IPV (Pediarix); DTaP-IPV/Hib (Pentacel), DTaP/Hib); pneumococcal (e.g. PCV13 (Prevnar13), PPSV23 (Pneumovax 23)); polio (e.g. (Ipol), DTaP-IPV (Kinrix), DTaP-HepB-IPV (Pediarix), DTaP-IPV/Hib (Pentacel)); rabies (Imovax Rabies, RabAvert); rotavirus RV1 (Rotarix), RV5 (RotaTeq); rubella (e.g. MMR (M-M-R II), MMRV (ProQuad)); shingles (Herpes Zoster) (e.g. ZOS (Zostavax)); smallpox (Vaccinia (ACAM2000)); tetanus (DTaP (Daptacel, Infanrix), Td (Decavac, generic), DT (generic), TT (generic), Tdap (Boostrix, Adacel), DTaP-IPV (Kinrix), DTaP-HepB-IPV (Pediarix), DTaP-IPV/Hib (Pentacel), DTaP/Hib); tuberculosis (TB) (e.g. BCG (TICE BCG, Mycobax)); typhoid (e.g. Typhoid Oral (Vivotif),Typhoid Polysaccharide (Typhim Vi)); or yellow fever (e.g. YF (YF-Vax)).

An immunological adjuvant is an agent that stimulates the immune system/increase the response to a vaccine or antigen, without having any specific antigenic effect in itself Immunological adjuvants are widely-known in the art, and include alum, including aluminum phosphate and aluminum hydroxide, QS-21 and squalene. A candidate immunological adjuvant is a potential immunological adjuvant in that it shows one or more biological properties of an immunological adjuvant, or properties described herein, and can subsequently be confirmed as an immunological adjuvant by testing in vivo.

This invention also provides a composition comprising an agent identified by one of the methods described herein as an immunological adjuvant, and a pharmaceutical carrier.

This invention also provides a method of making a vaccine comprising admixing a vaccine component as described herein with a pyroptosis inducer. In an embodiment, the method further comprises mixing with a pharmaceutically acceptable carrier. In an embodiment, the pyroptosis inducer comprises ATP, Bz-ATP, or nigericin.

Pharmaceutically acceptable carriers are preferably compatible with the adjuvant or adjuvant and vaccine compositions, and not significantly deleterious to the subject. Examples of acceptable pharmaceutical carriers include liposomes (which may encapsulate the aptamer-antigen conjugate, or which may be attached the aptamer-antigen conjugate) saline, carboxymethylcellulose, crystalline cellulose, glycerin, gum arabic, lactose, magnesium stearate, methylcellulose, powders, saline, sodium alginate, sucrose, starch, talc, and water, among others. Formulations of the pharmaceutical composition may conveniently be presented in unit dosage and may be prepared by any method known in the pharmaceutical art. For example, the aptamer, or aptamer conjugate or aptamer-liposome composition may be brought into association with a carrier or diluent, as a suspension or solution. Optionally, one or more accessory ingredients, such as buffers, flavoring agents, surface-active ingredients, and the like, may also be added. The choice of carriers will depend on the method of administration. The pharmaceutical composition can be formulated for administration by any method known in the art, including but not limited to, intravenously and orally.

The term “antigen” means all, or parts, of a protein, polypeptide, peptide or carbohydrate, and/or vaccine capable of causing an immune response in a vertebrate, preferably a mammal In an embodiment, the antigen is a protein, polypeptide or peptide. In a further embodiment, the protein, polypeptide or peptide may be glycosylated. In an embodiment, the antigen is a vaccine molecule. A “vaccine” as used herein is a chemical entity, capable of eliciting an immune response in an animal, preferably a mammal, when administered thereto as a vaccine. In non-limiting examples, the vaccine is an intact but inactivated (non-infective) or attenuated form of a biological pathogen, a purified or isolated component of a biological pathogen that is immunogenic (e.g., an outer coat protein of a virus), a toxoids (e.g. a modified tetanospasmin toxin of tetanus which is non-toxic itself).

In an embodiment, the immune response is a Th1 response. In an embodiment, the immune response is an adaptive immunity response. In an embodiment, the composition is administered in an amount sufficient to induce cytokine release by dendritic cells.

As used herein “and/or”, for example as in option A and/or option B, means the following embodiments: (i) option A, (ii) option B, and (iii) the option A plus B, and any subset of such options, including only one of the options.

The subject may be any subject. Preferably, the subject is a mammal More preferably, the subject is a human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

### Introduction

Analyzed herein is the role of lysosome rupture in Nlrp3 signaling, and it was found that disruption of lysosomes mediated by the adjuvants alum and LLOMe resulted in the release of lysosomal proteases (cathepsins) causing the degradation of multiple cellular proteins. Intriguingly, the inflammasome component caspase-1 and the caspase-1-associated cytokines IL-18 and IL-1β were among the most strongly degraded proteins. The proteolysis was independent of autocatalytic activities of the inflammasome complex, but highly dependent on the presence of specific cathepsins. In contrast, prototypical Nlrp3 inducers such as LPS and ATP strongly activated caspase-1 in vitro and in vivo, without induction of lysosome rupture and degradation of inflammatory proteins. Strikingly, both sets of compounds induced necrotic cell death with equal efficiency. Together, the data indicate that lysosome-disrupting agents and potassium efflux inducers differ drastically in their ability to activate Nlrp3, which might account for the different immune responses associated with these inducers.

### Materials and Methods

Chemicals and Reagents. Imject Alum was purchased from Thermo Scientific. Cell culture reagents were purchased from Fisher Scientific. Boc-D-CMK and Leu-Leu-OMe were purchased from Bachem (Torrance, Calif.). Propidium Iodide, ATP, and LPS (0111:B4) were purchased from Sigma-Aldrich (St. Louis, Mo.). Nigericin was purchased from EMD Chemical. LT was purchased from Wadsworth Laboratories. Cytotox One was purchased from Promega (Madison, Wis.). Precast gels and Comassie solutions were purchased from Biorad (Hercules, Calif.). Low-endotoxin fetal calf serum was purchased from Atlanta Biologicals (Norcross, Ga.).

Generation of primary cell lines and cell culture. Wild-type BALB/c and C57BL/6 mice were purchased from Jackson labs. Inflammasome deficient mice were provided by Dr. Fayyaz Sutterwala and were generated as described (39). Cathepsin-deficient mice were provided by Drs. Johanna Joyce and Thomas Reinheckel and were generated as described previously (40). All mice were euthanized humanly using CO₂ in compliance with standard protocols. Primary macrophages were generated from bone marrow from femurs and tibias as described (41, 42). In short, bone marrow was flushed from the femurs and tibias of mice under one year of age. Marrow was grown for one week in complete DMEM modified with 10% FCS, 20% L929 preconditioned media, 1% HEPES, 1% MEM non-essential amino acids, 0.1% cell-culture grade BME, 2% Pen/Strep. Marrow was grown for 6 days, at which point adherent cells were stripped and replated for assays. Cells were plated at 10⁶ cells/ml, except as needed for Western blotting (see below) in a solution of complete DMEM modified with 10% FCS, 10% L929 preconditioned media, 1% HEPES, 1% MEM non-essential amino acids, 0.1% cell-culture grade BME, 2% Pen/Strep. Cells were used within 4 days and then discarded.

Cell death assays and ELISAs. Necrosis was assessed by two methods: LDH release assays and propidium iodide (PI) exclusion. PI exclusion assays were performed in a 96-well flat-bottom plate using phenol-red-free DMEM and added to wells to a final concentration 30 μM 10-min before the specified time point. Fluorescence was measured using a Victor 2 plate reader from Perkin Elmer. LDH release was determined using the CytotoxOne kit from Promega, according to the manufacturer's instructions. In short, at specified time points, cells were pelleted in their wells at 500 g for 5 min, and 30-50 μl of supernatant was removed and mixed with an equal volume of CytotoxOne reagent. The enzymatic assay was developed until signal was obtained from a positive control (300 μM hydrogen peroxide) and the reaction was stopped and measured on a Victor 2 plate reader. IL-1β Ready-Set-Go ELISA kits were purchased from eBioscience and were performed according to manufacturer's recommendations. IL-1β concentration was determined at 1:1 and 1:10 to ensure linear range. All ELISA measurements were in triplicate and representative of 3 or more experiments. ELISAs were read on a Victor 2 plate reader from Perkin Elmer.

Western Blotting and Coomassie Staining. Macrophages were plated either on 24-well plates at 5×10⁵/well or in 6 well plates at 3×10⁶ cells/well to generate matched lysates and supernatants. Cell supernatants were collected from well plates and spun down at 300 g for 10 min at 4° C. 100 μl aliquots from spun samples were aliquoted into separate microfuge tubes with an equal volume of SDS sample buffer. For cell lysate preparations, RIPA buffer (Boston Bioproducts, Worcester, Mass.) containing protease inhibitor cocktail (Roche Applied Science, Indianapolis, Ind.) was added to cells in a 24 well plate and allowed to sit at 4° C. for 10 min. Cell lysates were collected and spun at 13,000 rpm for 10 min at 4° C. 100 μl of lysate was aliquoted into a separate microfuge tube with an equal volume of SDS sample buffer. Supernatant and lysate samples were placed in water bath at 100° C. for three min. Samples were normalized for total protein content using a Bradford assay, and then were run on 12% Tris-HCl gels (Biorad, Hercules, Calif.). Coomassie gels were immediately placed in Coomassie solution (Biorad) and allowed to stain overnight. Gels for westerns were then blotted onto PVDF membranes with a semi-dry transfer (Biorad). Membranes were probed with the following antibodies: anti-caspase-1 (Santa Cruz Biotechnologies, Santa Cruz, Calif.), anti-actin (Sigma-Aldrich, St. Louis, Mo.), anti-IL 18 (BioVision, Mountain View, Calif.), anti-IL-1β (R and D Systems Antibodies, Minneapolis, Minn.), anti-cathepsin B (R and D Systems Antibodies), and anti-cytochrome C (BD Biosciences, San Diego, Calif.). All secondary antibodies were HRP conjugated. Antibodies against goat, rabbit, and donkey were obtained from Santa Cruz. Anti-mouse secondary antibody was purchased from Amersham Biosciences (Piscataway, N.J.). Membranes were developed using Amersham ECL Plus solution.

Cathepsin Activity Assay. BALB/c macrophages were plated at 5×10⁵ per well in 24-well plates in triplicate and incubated with varying doses of Boc-D-CMK for 2 hours. Cathepsin activity was then measured as described previously (43). In short, cells were lysed in non-denaturing 50 mM MES buffer solution pH 5.5 solution with 0.05% TritonX 100, 135 mM NaCl, 2 mM EDTA at 4° C. Cell membranes were spun out at 16000 G for 5 min and post-nuclear supernatants were assayed for cathepsin activity. Cathepsin B and L activity were measured using Z-RR-AMC and Z-FR₂-AMC respectively (EMD Chemical, Gibbstown, Ni).

### Experimental Results

Lysosome-disrupting agents induce potent cell death but poor IL-1β release. A goal of these experiments was to determine the correlation between lysosome destabilization and Nlrp3 signaling. In order to test the hypothesis that lysosome rupture controls Nlrp3 inflammasome activation, the efficiency of Nlrp3 signaling mediated by lysosome-disrupting agents was compared with the potassium-efflux inducing agents prototypically associated with Nlrp3 activation (26, 44). Towards this, bone marrow-derived murine macrophages were challenged with both lysosome disrupting and pyroptosis-inducing agents and measured the amount of IL-1β and LDH released into the supernatant as complementary read-outs for Nlrp3 activation. To trigger lysosome disruption, alum and the dipeptide methyl ester, Leu-Leu-OMe (LLOMe), were used, both of which have been repeatedly shown to trigger lysosomal impairment (33, 45). As prototypical Nlrp3 inducers, the potassium-efflux-inducing agents ATP and nigericin were used in combination with LPS.

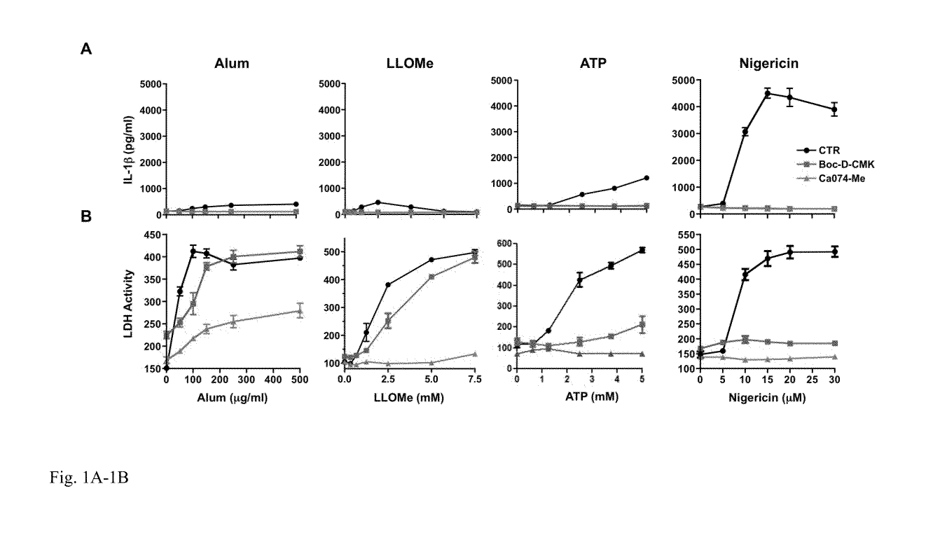

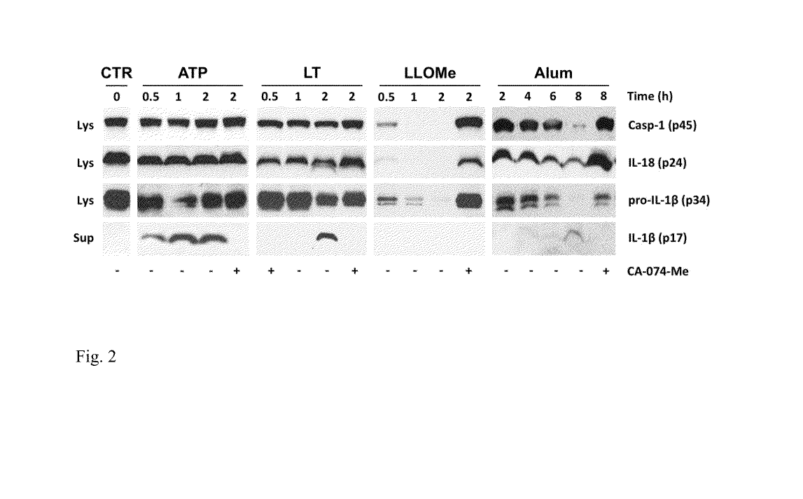

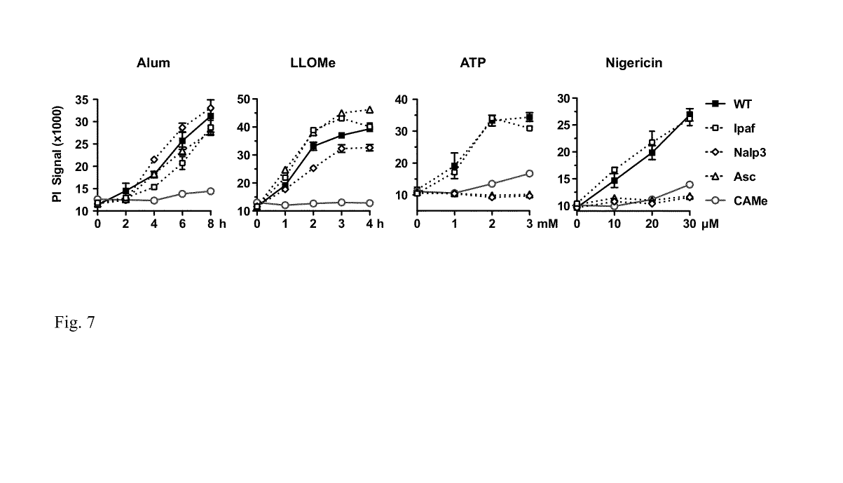

Nlrp3 activation results in two distinct caspase-1-mediated processes: IL-1β release and necrotic cell death (5). Here both processes were examined in parallel. Although lysosome disruption has been implicated in Nlrp3 inflammasome activation (46), it was found that the lysosome-disrupting agents alum and LLOMe triggered only minimal IL-1β release in murine macrophages (FIG. 1A). In contrast, the prototypical Nlrp3 inducers, nigericin (potassium ionophore) and ATP (activator of the potassium channel P2X7) (38, 44, 47, 48), triggered significant IL-1β release in LPS-stimulated murine macrophages (FIG. 1A). However, the caspase-1 inhibitor, Boc-D-CMK, blocked IL-1β secretion by all inducers tested, suggesting caspase-1-dependent IL-1β release (FIG. 1A). While Boc-D-CMK prevented cell death mediated by ATP and nigericin, it had no impact on alum and LLOMe-induced necrotic cell death (FIG. 1B). In contrast, the cathepsin B inhibitor, CA-074-Me, blocked not only IL-1β release, but also necrotic cell death mediated by all agents tested. Together this data suggested that ATP and nigericin triggered a distinctly different process of cell death and IL-1β release than alum and LLOMe. (Also see FIG. 7 and description thereof).

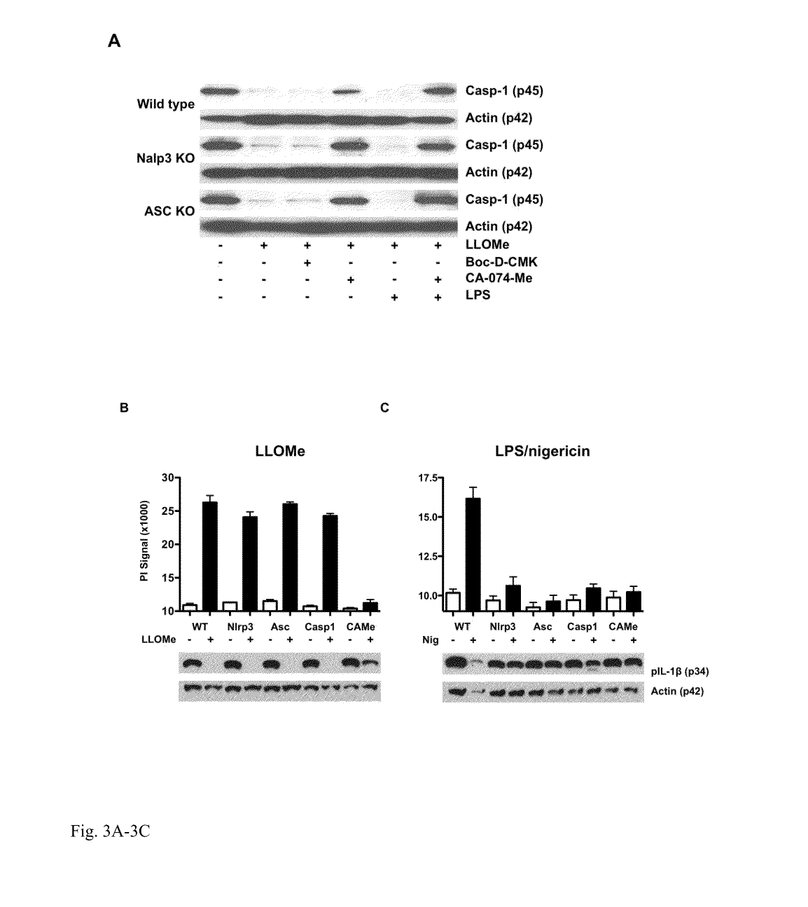

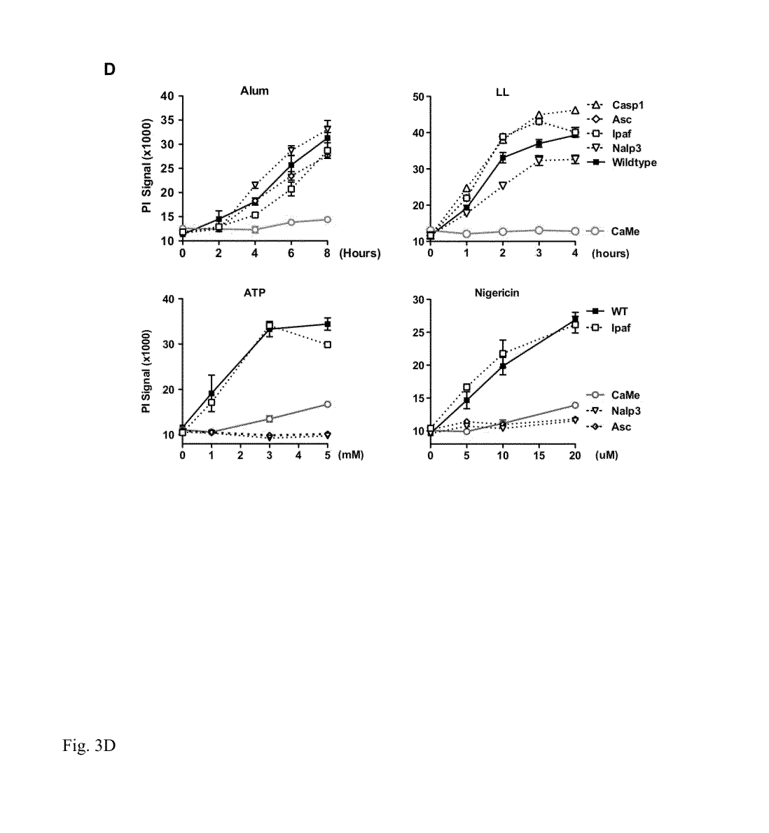

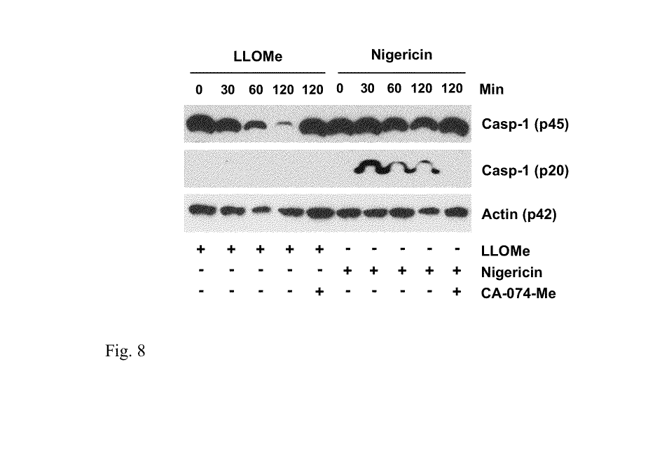

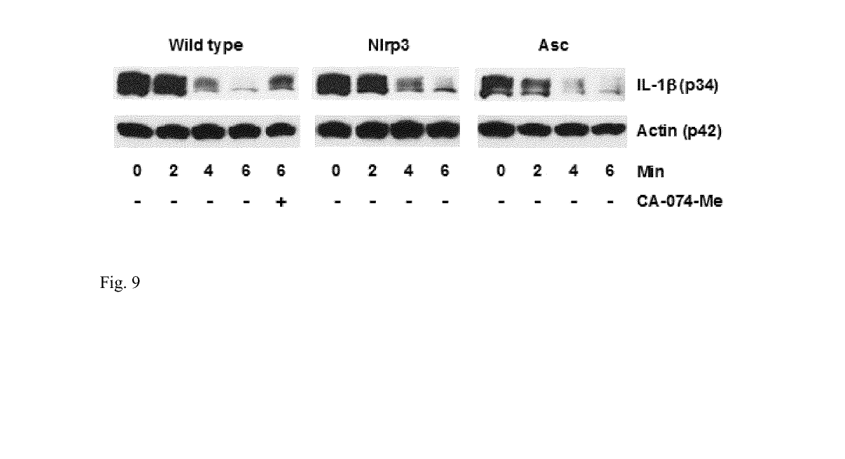

Alum and LLOMe-mediated depletion of caspase-1-associated proteins is independent of the Nlrp3 inflammasome. As a more direct way to assess Nlrp3 activation and cytokine activation by lysosome-disrupting agents and prototypical NLR inducers, activation of proinflammatory proteins was analyzed by immunoblotting. As expected, ATP (Nlrp3 inducer) and anthrax lethal toxin (LT: Nlrp1b inducer) strongly triggered the release of mature IL-1β into the supernatant of LPS-treated murine macrophages (FIG. 2). Consistent with the ELISA data (FIG. 1), only minimal or no mature IL-1β was found in the supernatant of alum or LLOMe-treated macrophages, respectively (FIG. 2). Intriguingly, alum and LLOMe triggered a dramatic decrease in cytosolic levels of IL-18, and IL-1β, which was not accompanied by apparent increase of these mature forms, suggesting that the pro-forms of these proteins were depleted (FIG. 2). As with IL-1β release, CA-074-Me prevented the drop in these proinflammatory proteins mediated by alum and LLOMe (FIG. 2). Intriguingly, alum and LLOMe treatment also resulted in a significant depletion of cellular pro-caspase-1 levels (FIGS. 2 and 3A). Because autocatalytic processing is required for caspase-1-activation, it was investigated whether autocatalysis of caspase-1 was involved in this process. Processing of pro-caspase-1 (p45) into an active dimer of p10 and p20 isoforms was assessed by immunoblotting (FIG. 3A). It was found that, as is typical for Nlrp3 inducers, ATP and nigericin triggered minimal decrease of p45 with a notable increase of p20 or p10 (FIG. 2). However, in a strikingly different behavior, LLOMe and alum triggered a substantial depletion of p45 without generating the p20 or p10 subunits (FIG. 2). Together these results suggested that lysosome disruption by alum and LLOMe leads to the depletion of inflammasome-associated proteins without significant caspase-1 activation. No mature caspase-1 (p20) indicative of caspase-1 activation in LLOMe-treated macrophages was found. (See FIG. 8 and description thereof).

As inflammasome activation involves proteolytic processing of caspase-1, IL-18, and IL-1β it was then tested whether autocatalytic processes contributed to the drop pro-caspase-1, pro-IL-18, and pro-IL-1β in alum and LLOMe-treated macrophages. It was found that Nlrp3 or Asc-deficiency had no impact on the LLOMe-mediated drop in pro-caspase-1 and IL-1β levels (FIG. 3B). Similar results were obtained with alum suggesting that the observed decrease in proinflammatory proteins was independent of Nlrp3 signaling. Nlrp3 or Asc-deficiency also failed to prevent LLOMe-mediated cell death, indicating inflammasome-independent cell death (FIGS. 3B and D). In contrast, Nlrp3, Asc, and caspase-1-deficiency prevented cell death and IL-1β processing mediated by nigericin consistent with inflammasome-mediated necrosis (FIG. 3C). Nlrp3, Asc, and caspase-1-deficiency also prevented the drop in pro-IL18 in nigericin-treated macrophages consistent with an inflammasome-controlled process (FIG. 3C). Intriguingly, CA-074-Me blocked necrosis by all inducers tested (FIG. 3). CA-074-Me also blocked alum and LLOMe-mediated protein degradation (FIG. 3). Taken together, the findings indicate that alum and LLOMe-mediated depletion of inflammasome-associated proteins occurred independently of Nlrp3 signaling. The findings suggested fundamental differences between inflammasome activation mediated by the lysosome-disrupting agents and the pyroptosis inducers.

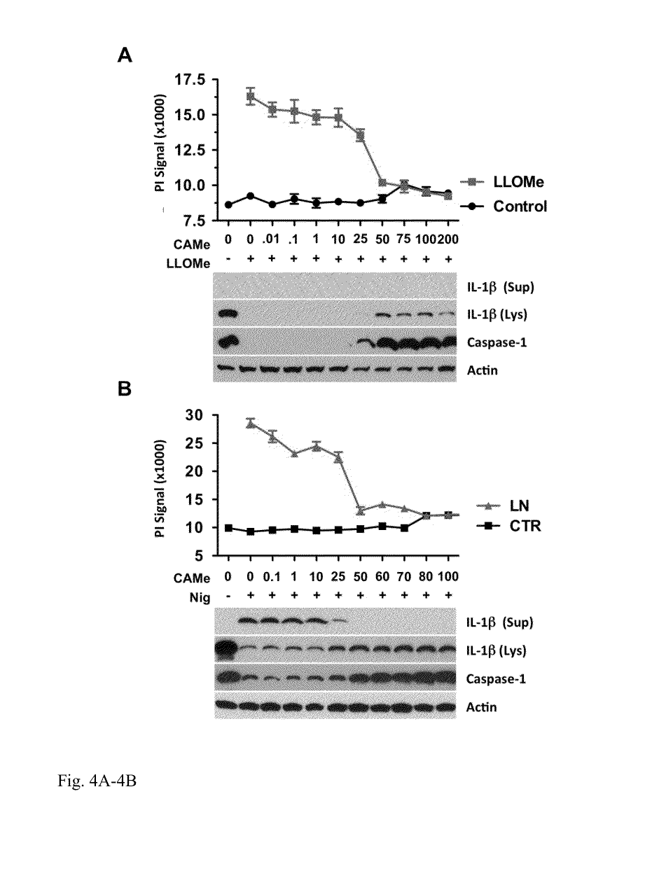

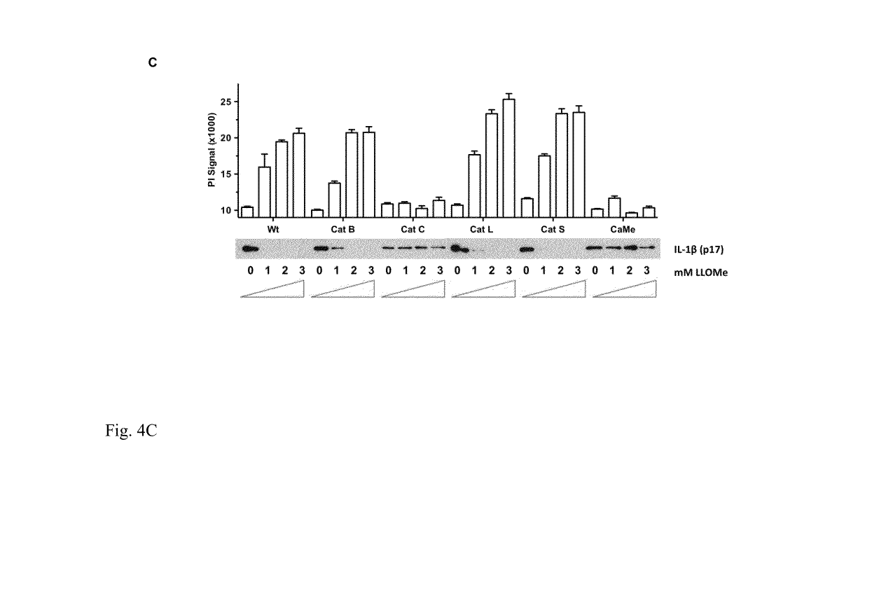

Next analyzed was the correlation between cell death and degradation of inflammatory proteins in alum- and LLOMe-mediated macrophages. It was found that increasing CA-074-Me concentrations blocked both, cell death and the drop in inflammatory proteins, in LLOMe-treated macrophages (FIG. 4A). In fact, levels of inflammatory proteins correlated perfectly with cell death induction in LLOMe-treated macrophages (FIG. 4A). Very different results were obtained in nigericin-treated macrophages (FIG. 4B). Nigericin triggered caspase-1 activation and Nlrp3 signaling as indicated by the appearance of mature IL-1β in the supernatant (FIG. 4B). ATP-treated cells behaved identically to nigericin (data not shown). CA-074-Me concentrations that blocked cell death by these Nlrp3 inducers also prevented the release of processed IL-1β (FIG. 4B). Increasing CA-074-Me concentrations resulted in an increasing reduction of cytokine processing and cell death induction indicating a perfect correlation between IL-1β processing (Nlrp3 signaling) and cell death induction, as expected from a pyroptosis inducer. The reduction in pro-IL-1β was concurrent with the appearance of mature IL-1β in the supernatant, consistent with cytokine processing, but not protein degradation in these cells. Taken together, the findings indicated a perfect correlation between LLOMe-induced cell death and the depletion of inflammatory proteins.

Alum and LLOMe-mediated inflammasome degradation is dependent on the activity of specific cathepsins. A perfect correlation between cell death and protein degradation in alum and LLOMe-treated macrophages (FIG. 3) has been demonstrated here. Previously, it has been demonstrated that specific cathepsins control cell death mediated by these lysosome-disrupting agents. It was next asked whether the same cathepsins that control alum and LLOMe-induced cell death were critical for the degradation of inflammasome components. While having previously established that cathepsin C is critical for LLOMe-mediated cell death, as predicted, cathepsin C deficiency prevented the degradation of the inflammatory protein IL-1β, as well as cell death. Cathepsin B, L and S deficiency had no impact on protein degradation and cell death indicating the specificity of this process (FIG. 4C). It was previously shown that cathepsin B-deficiency has no impact on LLOMe-mediated cell death, but it impairs alum-mediated cell death. Accordingly, a significant reduction in cell death and caspase-1 degradation was found in cathepsin B-deficient macrophages following alum exposure, while cathepsin C-deficiency had no impact on these processes. Together, these data suggest that specific cathepsins control cell death and degradation of inflammatory proteins by lysosome-disrupting agents.

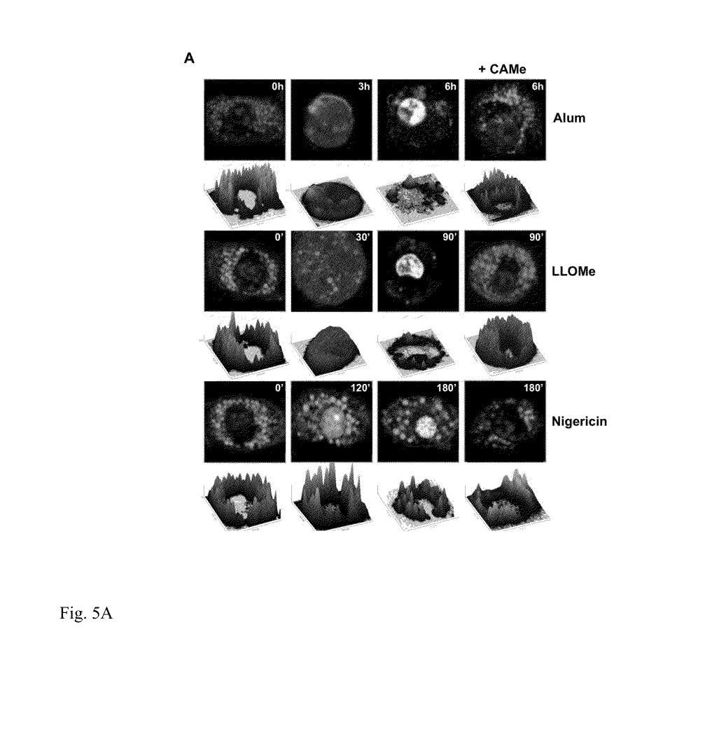

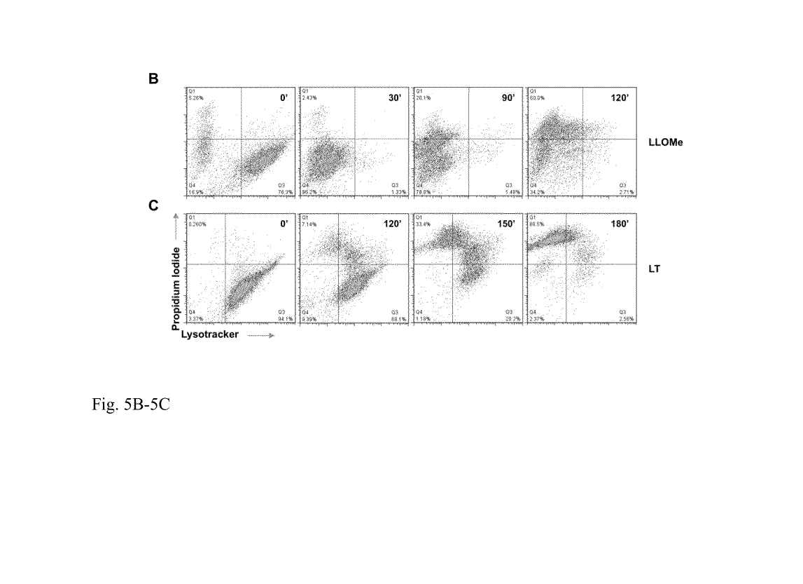

Cathepsin-dependent lysosome rupture correlates with degradation of inflammatory proteins. Previous studies had suggested that lysosome disruption is a critical step for Nlrp3 activation by a number of compounds. To this point it has been demonstrated that lysosome-disrupting agents act distinctly from known pyroptosis inducers. Lysosome-disrupting agents caused caspase-1-independent cell death and broad protein degradation, while pyroptosis inducers caused caspase-1-dependent cell death and targeted IL-1β secretion. In order to directly investigate the role of lysosome rupture in Nlrp3 activation, two complementary methods were used to analyze lysosome integrity in macrophages challenged with both lysosome-destabilizing agents and potassium-efflux inducers. First, lysosomal integrity was measured by labeling vesicles in the endolysosomal pathway with fluorescent dextran. It was found that fluorescent-dextran progressed from the punctate lysosomal staining observed in untreated cells to a diffuse cytosolic and nuclear pattern within 3-4 hours of alum exposure suggesting induction of lysosome rupture (FIG. 5A). Intriguingly, no plasma membrane impairment (indicative of necrotic cell death) was detectable at this time point, but was observed only 6 hours post alum exposure (FIG. 5A). Similar results were obtained with LLOMe, though with faster kinetics (FIG. 5A). LLOMe destabilized lysosomes within 30 min, without impairment of plasma membranes. A loss of plasma membrane integrity was observed only after 60 min of LLOMe exposure, resulting in dextran release from these cells (FIG. 5A). CA-074-Me treatment blocked lysosomal release of dextran from alum and LLOMe-treated macrophages suggesting that cathepsin inhibitors block necrosis by preventing lysosome rupture. In contrast to alum and LLOMe, the pyroptosis inducers, LPS and nigericin, did not trigger any discernable lysosome rupture prior to a loss of plasma membrane integrity. In fact, LPS/nigericin-treated macrophages were completely necrotic by 2 hours, but showed no signs of lysosome rupture (FIG. 5A).

To quantify these findings, lysosomal integrity was also analyzed by flow cytometry with the lysosomal pH-indicator LysoTracker and the vital stain propidium iodide (FIG. 5B and C). It was found that LLOMe triggered a complete loss of lysosome integrity within 30 min as observed microscopically, while necrotic cell death was only observed after 60-90 min of LLOMe treatment (FIG. 5B). Assessing lysosome integrity by LysosoTracker in nigericin-treated cells proved impractical as nigericin non-specifically quenches the LysoTracker signal in the absence of lysosomal impairment. Intriguingly, protein degradation was concurrent with a loss of lysosome integrity 30 to 45 min after LLOMe exposure, and 4-6 hours after alum exposure (FIGS. 5A and 5B). By contrast, lysosome impairment was a late event in nigericin and LT-treated cells, and occurred only after cell death induction (FIGS. 5A and 5C). Taken together, these findings demonstrate the fundamental differences between necrotic cell death mediated by lysosome-disrupting agents and pyroptosis inducers. These findings suggested that lysosomal release of proteolytic enzymes, such as cathepsins, is critical for protein degradation observed in these cells.

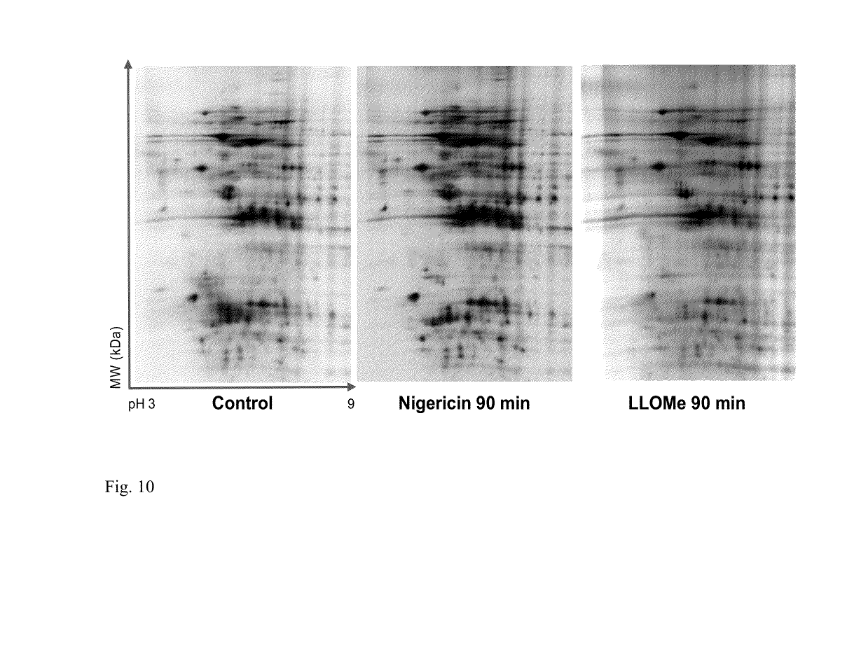

Broad protein degradation mediated by LLOMe—As lysosomal proteases are highly promiscuous, it was reasoned that alum- and LL-mediated lysosomal release of proteolytic proteins may not just degrade Nlrp3-associated proteins, but may also trigger degradation of a broad range of cytosolic proteins, and might go beyond the degradation of inflammatory proteins reported here. To examine effects of LLOMe on the cellular proteome, macrophages were challenged with these compounds and generated lysates. 2D-DIGE was used to determine the extent of damage inflicted in macrophages exposed to the lysosome-disrupting agent, LLOMe. As predicted, it was found that LLOMe triggered broad degradation of proteins within these cells, visible on a macro-level by 2D-DIGE (FIG. 10). As a control the Nlrp3/pyroptosis inducer, nigericin, was used which did not trigger any apparent degradation of cellular proteins (FIG. 10). These findings further highlight the different impact of lysosome-destabilizing agents and pyroptosis inducers on the cellular proteome on a macro-level.

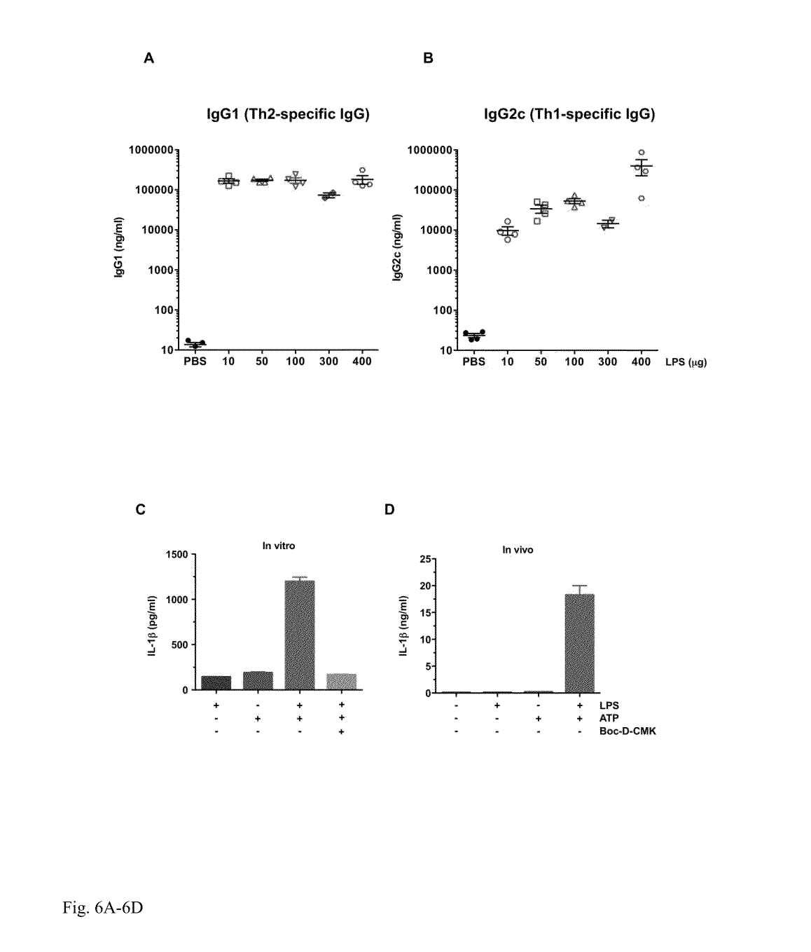

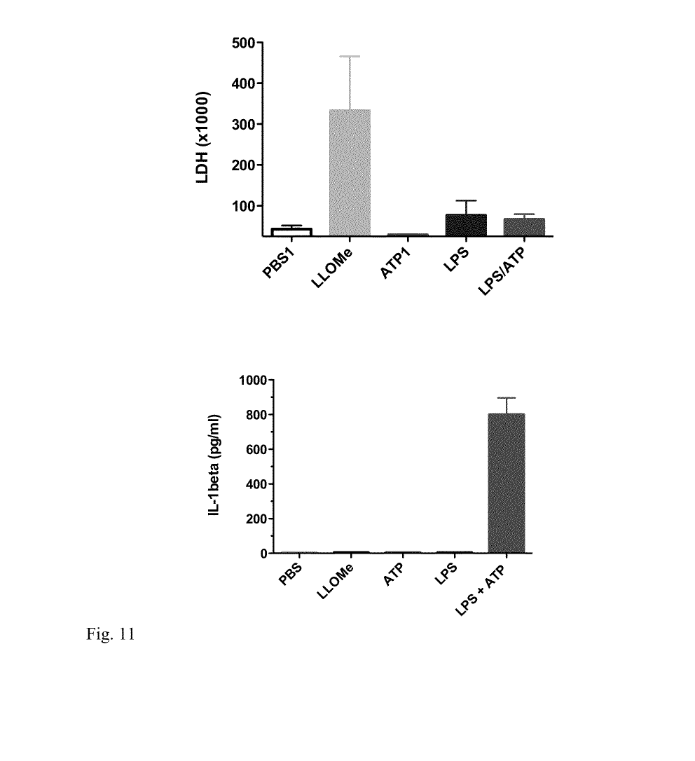

Prototypical Nlrp3 inducers trigger a strong IL-1β in vivo and production of Th1-specific antibodies in vivo. While the substantial difference in Nlrp3-signalling in vitro is shown, it was further chosen to investigate the ability of lysosome-disrupting agents and pyroptosis inducer to activate Nlrp3 in vivo. A combination of LPS and ATP has been suggested to mimic the effects of LPS alone in vivo (49). High concentrations of LPS have been shown to trigger the autocrine release of ATP from monocytes, which activates the potassium channel P2X7 and presumably provides the necessary secondary signal for Nlrp3 (FIG. 2). To this end, the effects of LPS, LLOMe, and alum on IL-1β signaling and LDH release were compared in vivo. Intriguingly, it was found that only minimal IL-1β or LDH release occurred in mice following exposure to high concentrations of LPS. It was possible, however, to overcome this by adding the exogenous potassium-efflux inducer ATP to the cocktail. Strikingly, the presence of ATP not only drastically enhanced Nlrp3 in LPS-treated macrophages, but it also substantially increased IL-1β release in vivo when used in conjunction with LPS (FIGS. 6C and D). It was found that C57BL/6 mice triggered significant IL-1β after intraperitoneal priming with low concentrations of LPS, followed by i.p. challenge with 100 mM ATP (FIG. 6C). Similarly, cell death was substantially increased in these mice as well, as indicated by elevated LDH levels. Consistent with the in vitro results, alum and LLOMe triggered significant release of LDH when injected intraperitoneally into mice, but triggered poor release of IL-1β. Consistent with in vitro results, LLOMe triggered significant release of LDH when injected intraperitoneally into mice, but triggered poor release of IL-1β (FIG. 11).

Intriguingly, LPS, alum, and LLOMe exhibit all adjuvant activity. While all have been suggested to act through Nlrp3 on an in vitro level, it is interesting that while alum and LLOMe are polarized Th2-inducing adjuvants, LPS is a prototypical Th1-inducing adjuvant. It was sought to determine if this difference correlated with the observed difference in in vivo IL-1β activation. To test this hypothesis, C57BL/6 mice were immunized with alum, LLOMe, and LPS. It was found that LPS triggered a strong IgG2c response in a dose-dependent fashion in mice, suggestive of a Th1 response (FIG. 6). Intriguingly, LPS also induced a Th2 response in a dose-independent fashion (FIG. 6). By contrast, alum and LLOMe triggered an immune response with polarized IgG1, and little induction of IgG2c. Taken together, these findings are strongly suggestive that the difference in Nlrp3 activation in vivo may contribute to the differences in immunologic function between both agents.

### Discussion

Lysosome disruption is not a critical regulator of Nlrp3 activation. A wide variety of substances activate Nlrp3 without direct interaction with this receptor. The chemical and structural unrelatedness of Nlrp3 inducers suggests that they trigger secondary event(s) critical for Nlrp3 activation. To determine whether lysosome rupture could activate Nlrp3, lysosome rupture was correlated with inflammasome signaling mediated by a range of Nlrp3 inducers. While lysosome rupture has been suggested to be a potent activator of Nlrp3, it was found that lysosome-disrupting compounds differ fundamentally from potassium-efflux generating agents in their ability to trigger lysosome rupture and to activate the Nlrp3 inflammasome. It was also found that the lysosome-destabilizing agents, alum and LLOMe, triggered only minimal IL-1β processing compared to levels induced by pore-forming toxins. While they initially triggered a small amount of caspase-1-dependent IL-1β release, this appears to be quickly overwhelmed by rapid degradation of caspase-1 and IL-1β. Minimal Nlrp3 signaling by lysosome-disrupting agents was surprising because lysosome rupture has been implicated in Nlrp3 signaling. In fact, it was found that alum and LLOMe triggered a sharp decrease in the proforms of the Nlrp3-associated proteins caspase-1, IL-18, and IL-1β. The depletion of the inflammasome component caspase-1 was independent of Nlrp3 signaling, and not associated with a corresponding increase in the mature form. This finding indicates that the alum and LLOMe-induced decrease in caspase-1 was not due to autocatalytic processes, but caused by other caspase-1-independent proteolytic processes. Taken together these findings strongly suggest that lysosome disruption does not control Nlrp3. These findings do not, however, contradict a model in which a common upstream event controls Nlrp3 activation, they only indicate that lysosome rupture is not this event.

Lysosome rupture and lysosomal cathepsins control degradation of inflammatory proteins. It was found that degradation of pro-caspase-1, IL-18, and IL-1βoccurred when lysosomal contents were released into the cytosol prior to plasma membrane impairment, and were therefore constrained within the cell. In the case of alum and LLOMe, processes that controlled cell death and protein degradation could not be separated, suggesting that these two were related processes. Agents that blocked alum and LLOMe-mediated cell death, such as the cathepsin B inhibitor CA-074-Me, also prevented the degradation of inflammatory proteins caused by alum and LLOMe. A specific cathepsin (cat C) has previously been identified that is critical for cell death and adjuvant activities mediated by LLOMe. Intriguingly, cathepsin C-deficiency also prevented LLOMe-induced degradation of proinflammatory proteins. Moreover, alum and LLOMe-induced lysosome rupture coincided with degradation of proinflammatory proteins, and preceded necrotic cell death.

Based on these findings, a model has been proposed in which the cytosolic constrainment of lysosomal proteases results in the degradation of proinflammatory proteins observed in alum and LLOMe-treated macrophages. Lysosomes contain a number of enzymes including lipases, proteases, amylases, and nucleases that are used to digest foreign bodies and to recycle cellular components (51, 52). Many of these enzymes are highly promiscuous and are primarily regulated by compartmentalization (53). Based on the findings, it is considered that when alum and LLOMe-mediated lysosome rupture results in the release of these proteolytic enzymes into the cytosol, these enzymes induce degradation of cytosolic proteins, and possibly plasma membrane rupture. Because these hydrolases were released into the cytosol significantly before plasma membrane impairment, the released lysosomal proteases were retained within the cytosol during this early phase in alum and LLOMe-treated macrophages. This is consistent with previous studies indicating that lysosomal acid hydrolases, such as cathepsins, can retain their activity even after release into the neutral-pH of the cytosol (54-56). By contrast, ATP and nigericin, whose lysosomal rupture did not occur until significantly after plasma membrane impairment, showed minimal protein degradation. It is therefore conceivable that the constrainment of lysosomal contents into the cytosol enhances the destructive potential of lysosomal hydrolases.

The broad release of cathepsins into the cytosol is suggestive of a non-specific proteolytic process. Taken together, these findings suggest that the ingestion of lysosome-disrupting agents triggers protein degradation and necrotic cell death that is distinct from known processes. As many insoluble particles, including alum, silica, asbestos, cholesterol plaques, and ultra-high molecular weight polyethylene have been suggested to cause both, lysosomal rupture and systemic disease, it is conceivable that this broad destructive process observed with alum and LLOMe may also contribute to their pathology. However, further study is needed to determine if the findings can be extended to these agents.

Nlrp3 activation correlates with the production of Th1-associated antibodies. It has been demonstrated herein that lysosome-disrupting agents, such as alum and LLOMe, neutralize the Nlrp3 inflammasome complex. This is consistent with the findings that alum and LLOMe trigger only minimal IL-1β release/caspase-1 activation in macrophages, as well as in vivo. Recent studies have implicated Nlrp3 signaling in alum-mediated adjuvant effects (32, 35, 57). While the original studies have indicated Nlrp3-dependent antibody production in alum-treated mice, more recent studies suggested that caspase-1 and Nlrp3 are dispensable for alum-enhanced immune responses consistent with the findings (11, 12, 34, 57-62). Nevertheless, the findings do not rule out the possibility that minimal Nlrp3 activation might still contribute to alum-enhanced immunity. Nlrp3 contribution is less likely in the case of LLOMe, as this adjuvant almost completely obliterates the inflammasome with minimal or no signs of caspase-1 activation. Findings here show that necrotic cell death is a better correlate than Nlrp3 signaling for alum and LLOMe-mediated adjuvant effects. In a recent study, it has been demonstrated that LLOMe mimics alum in both, necrotic cell death mediated by specific cathepsins, and by its ability to trigger a Th2-specific immune response. Moreover, it has been demonstrated that cathepsin C deficiency impaired not only LLOMe-induced cell death, but also adjuvant effects associated with LLOMe. In fact, LLOMe was even an even more powerful adjuvant than alum. These findings are consistent with studies implicating the necrotic release of uric acid and DNA in alum's in vivo responses (60, 63). These studies indicated that blocking uric acid and DNA release prevents alum's adjuvant effects consistent with studies linking necrotic cell death with enhancement of the adaptive immune response (34, 59, 60, 64, 65). Taken together, the findings suggest that lysosome rupture antagonizes Nlrp3 signaling and promotes necrotic cell death, which appears to be a strong inducer of a Th2-biased immune response.

Several studies have indicated that the Nlrp3 inflammasome is a powerful trigger of the adaptive immune response in mice challenged with specific microbial pathogens. For example, it has been shown that Nlrp3 activates the adaptive immune response in mice challenged with diverse microbial pathogens, including Streptococcus pyogenes, Klebsiella pneumoniae, and Candida albicans [71,72,73]. Importantly, Nlrp3-deficient mice showed a greatly diminished protective immune response and survival following challenge with these pathogens consistent with a critical role of Nlrp3 in establishing an adaptive immune response against these pathogens. Based on these findings it has been suggested that adjuvants take advantage of this endogenous system that is able to promote a protective immune response. The findings here, however, suggest that the adjuvants alum (and LLOMe) trigger an Nlrp3-independent immune response. While these studies strongly argued against a role of Nlrp3 in alum's adjuvanticity, it was asked whether other agents with a better Nlrp3 signaling propensity could fill in this gap, and trigger an Nlrp3-dependent adaptive immune response.

In contrast to lysosome-destabilizing agents, the prototypical Nlrp3 inducers ATP and nigericin strongly induced IL-1β release in LPS-treated macrophages. As expected, these agents triggered no discernable degradation of the proteome, and especially of proinflammatory proteins. Also, no lysosome rupture was detected prior to inflammasome activation and cell death induction in cells exposed to these Nlrp3 inducers. In fact, lysosomes remained intact even after inflammasome activation and even after cells showed signs of necrosis. While these pyroptosis-inducers did not trigger any discernable early lysosome rupture, it cannot be ruled out that limited lysosomal release of cathepsins, presumably from intact lysosomes, is still involved in Nlrp3 signaling. It has been previously reported that LPS triggers the release of ATP from monocytes, which is a strong cosignal for Nlrp3 signaling. However, only minimal IL-1β release was found in mice following exposure to high concentrations of LPS. It was possible to overcome the limited IL-1β release by addition of exogenous ATP to the cocktail. Strikingly, the presence of ATP drastically enhanced Nlrp3 signaling in LPS-treated macrophages. In addition, it was demonstrated that the Gram-negative cell wall component, LPS, in contrast to alum and LLOMe, triggers a strong Th1 response in a dose-dependent fashion in mice. Intriguingly, LPS also induced a dose-independent Th2 response as well. This is in stark contrast to lysosome-destabilizing agents alum and LLOMe, which polarize the immune response towards a strong Th2 bias (66-68). Consistent with a role of Nlrp3 in eliciting an adaptive immune response, the Nlrp3-associated cytokines have been linked to a Th1 response (69-74). It is therefore very conceivable that Nlrp3 contributes to the adjuvant activity of LPS or other Nlrp3 inducers.

In summary, the data indicate fundamental differences between the lysosome-destabilizing agents alum/LLOMe and the pyroptosis-inducer LPS. It has been shown herein that these inducers differ significantly in the cell death pathways they trigger: i.e., lysosome-mediated cell death or caspase-1-mediated (pyroptosis), respectively. In addition, both inducers differ drastically in their ability to activate the Nlrp3 inflammasome. While pyroptosis-inducers trigger robust processing and release of IL-18 and IL-1β, the lysosome-destabilizing agents alum and LLOMe degrade these proteins resulting in minimal secretion. These lysosome-disrupting agents appear instead to antagonize Nlrp3 signaling. It is believed that these differences observed on a cellular level between these inducers can explain their drastically different immune responses they trigger in vivo. The findings suggest that strong activation of Nlrp3 in vivo by LPS induces an adaptive immune response. The studies further indicate that Nlrp3 activation by alum and LLOMe is too weak to drive an associated Nlrp3 immune response.

### References

1. Requires Streptolysin O and NF-kappa B Activation but Proceeds Independently of TLR Signaling and P2X7 Receptor. J Immunol 183(9):5823-5829.
2. Willingham S B, et al. (2009) NLRP3 (NALP3, Cryopyrin) facilitates in vivo caspase-1 activation, necrosis, and HMGB1 release via inflammasome-dependent and -independent pathways. J Immunol 183 (3):2008-2015.
3. Joly S, et al. (2009) Cutting edge: Candida albicans hyphae formation triggers activation of the Nlrp3 inflammasome. J Immunol 183(6):3578-3581.
4. Mariathasan S & Monack D M (2007) Inflammasome adaptors and sensors: intracellular regulators of infection and inflammation. Nature reviews. Immunology 7(1):31-40.
5. Franchi L, Eigenbrod T, Munoz-Planillo R, & Nunez G (2009) The inflammasome: a caspase-1-activation platform that regulates immune responses and disease pathogenesis. Nat Immunol 10(3):241-247.
6. Fantuzzi G & Dinarello C A (1999) Interleukin-18 and interleukin-1 beta: Two cytokine substrates for ICE (Caspase-1). J Clin Immunol 19(1):1-11.
7. Khoruts A, Osness R E, & Jenkins M K (2004) IL-1 acts on antigen-presenting cells to enhance the in vivo proliferation of antigen-stimulated naive CD4 T cells via a CD28-dependent mechanism that does not involve increased expression of CD28 ligands. Eur J Immunol 34(4):1085-1090.
8. Pollock K G, Conacher M, Wei X Q, Alexander J, & Brewer J M (2003) Interleukin-18 plays a role in both the alum-induced T helper 2 response and the T helper 1 response induced by alum-adsorbed interleukin-12. Immunology 108(2):137-143.
9. Nakae S, et al. (2001) IL-1 alpha, but not IL-1 beta, is required for contact-allergen-specific T cell activation during the sensitization phase in contact hypersensitivity. International immunology 13(12):1471-1478.
10. Schmitz J, et al. (2005) IL-33, an interleukin-1-like cytokine that signals via the IL-1 receptor-related protein ST2 and induces T helper type 2-associated cytokines. Immunity 23(5):479-490.
11. Franchi L & Nunez G (2008) The Nlrp3 inflammasome is critical for aluminium hydroxide-mediated IL-1beta secretion but dispensable for adjuvant activity. Eur J Immunol 38(8):2085-2089.
12. McKee A S, et al. (2009) Alum induces innate immune responses through macrophage and mast cell sensors, but these sensors are not required for alum to act as an adjuvant for specific immunity. J Immunol 183(7):4403-4414.
13. Kanneganti T-D, et al. (2006) Critical Role for Cryopyrin/Nalp3 in Activation of Caspase-1 in Response to Viral Infection and Double-stranded RNA. J Biol Chem 281(48):36560-36568.
14. Kanneganti T D, et al. (2006) Critical role for Cryopyrin/Nalp3 in activation of caspase-1 in response to viral infection and double-stranded RNA. J Biol Chem 281(48):36560-36568.
15. Hise A G, et al. (2009) An essential role for the NLRP3 inflammasome in host defense against the human fungal pathogen Candida albicans. Cell Host Microbe 5(5):487-497.
16. Petrilli V, Dostert C, Muruve D A, & Tschopp J (2007) The inflammasome: a danger sensing complex triggering innate immunity. Curr Opin Immunol 19(6):615-622.
17. Duncan J A, et al. (2009) Neisseria gonorrhoeae activates the proteinase cathepsin B to mediate the signaling activities of the NLRP3 and ASC-containing inflammasome. J Immunol 182(10):6460-6469.
18. Ichinohe T, Lee H K, Ogura Y, Flavell R, & Iwasaki A (2009) Inflammasome recognition of influenza virus is essential for adaptive immune responses. J Exp Med 206(1):79-87.
19. Abdul-Sater AA, Said-Sadier N, Padilla E V, & Ojcius D M (2010) Chlamydial infection of monocytes stimulates IL-1beta secretion through activation of the NLRP3 inflammasome. Microbes Infect 12(8-9):652-661.
20. Wong K-W & Jacobs Jr W R (2011) Critical role for NLRP3 in necrotic death triggered by Mycobacterium tuberculosis. Cell Microbiol 13(9): 1371-1384.
21. Willingham S B, et al. (2007) Microbial pathogen-induced necrotic cell death mediated by the inflammasome components CIAS1/cryopyrin/NLRP3 and ASC. Cell Host Microbe 2(3):147-159.
22. Kim S, et al. (2010) Listeria monocytogenes is sensed by the NLRP3 and AIM2 inflammasome. Eur J Immunol 40(6): 1545-1551.
23. Halle A, et al. (2008) The NALP3 inflammasome is involved in the innate immune response to amyloid-beta. Nat Immunol 9(8):857-865.
24. Franchi L, Warner N, Viani K, & Nunez G (2009) Function of Nod-like receptors in microbial recognition and host defense. Immunological reviews 227(1):106-128.
25. Kanneganti T D, et al. (2006) Bacterial RNA and small antiviral compounds activate caspase-1 through cryopyrin/Nalp3. Nature 440(7081):233-236.
26. Mariathasan S, et al. (2006) Cryopyrin activates the inflammasome in response to toxins and ATP. Nature 440(7081):228-232.
27. Martinon F (2008) Detection of immune danger signals by NALP3. J Leukoc Biol 83(3):507-511.
28. Martinon F, Agostini L, Meylan E, & Tschopp J (2004) Identification of bacterial muramyl dipeptide as activator of the NALP3/cryopyrin inflammasome. Curr Biol 14(21):1929-1934.
29. Martinon F, Petrilli V, Mayor A, Tardivel A, & Tschopp J (2006) Gout-associated uric acid crystals activate the NALP3 inflammasome. Nature 440(7081):237-241.
30. Sharp F A, et al. (2009) Uptake of particulate vaccine adjuvants by dendritic cells activates the NALP3 inflammasome. Proc Natl Acad Sci USA 106(3):870-875.
31. Gonzalez-Benitez J F, et al. (2008) The NALP3/Cryopyrin-inflammasome complex is expressed in LPS-induced ocular inflammation. Mediators Inflamm 2008: 614345.
32. Eisenbarth S, Colegio O, O'Connor W, Sutterwala F, & Flavell R (2008) Crucial role for the Nalp3 inflammasome in the immunostimulatory properties of aluminium adjuvants. Nature 453(7198):1122-1126.
33. Hornung V, et al. (2008) Silica crystals and aluminum salts activate the NALP3 inflammasome through phagosomal destabilization. Nat Immunol 9(8):847-856.
34. Kool M, et al. (2008) Cutting edge: alum adjuvant stimulates inflammatory dendritic cells through activation of the NALP3 inflammasome. J Immunol 181(6):3755-3759.
35. Li H, Willingham S B, Ting J P, & Re F (2008) Cutting edge: inflammasome activation by alum and alum's adjuvant effect are mediated by NLRP3. J Immunol 181(1):17-21.
36. Jin C C & Flavell R A (2010) Molecular Mechanism of NLRP3 Inflammasome Activation. J Clin Immunol 30(5):628-631.
37. Naik E & Dixit V M (2011) Mitochondrial reactive oxygen species drive proinflammatory cytokine production. J Exp Med 208(3):417-420.
38. Hentze H, Lin X Y, Choi M S, & Porter A G (2003) Critical role for cathepsin B in mediating caspase-1-dependent interleukin-18 maturation and caspase-l-independent necrosis triggered by the microbial toxin nigericin. Cell Death Differ 10(9):956-968.
39. Sutterwala F S, et al. (2006) Critical role for NALP3/CIAS1/Cryopyrin in innate and adaptive immunity through its regulation of caspase-1. Immunity 24(3):317-327.
40. Halangk W, et al. (2000) Role of cathepsin B in intracellular trypsinogen activation and the onset of acute pancreatitis. The Journal of clinical investigation 106(6):773-781.
41. Muehlbauer S M, et al. (2010) Proteasome inhibitors prevent caspase-1-mediated disease in rodents challenged with anthrax lethal toxin. Am J Pathol 177(2):735-743.
42. Muehlbauer S M, et al. (2007) Anthrax lethal toxin kills macrophages in a strain-specific manner by apoptosis or caspase-1-mediated necrosis. Cell Cycle 6(6):758-766.
43. Wong A C, Sandesara R G, Mulherkar N, Whelan S P, & Chandran K (2010) A Forward Genetic Strategy Reveals Destabilizing Mutations in the Ebolavirus Glycoprotein That Alter Its Protease Dependence during Cell Entry. J Virol 84(1):163-175.
44. Kahlenberg J M & Dubyak G R (2004) Mechanisms of caspase-1 activation by P2X7 receptor-mediated K+ release. American journal of physiology. Cell physiology 286(5):C1100-1108.
45. Thiele D L & Lipsky P E (1990) Mechanism of L-leucyl-L-leucine methyl ester-mediated killing of cytotoxic lymphocytes: dependence on a lysosomal thiol protease, dipeptidyl peptidase I, that is enriched in these cells. Proc Natl Acad Sci USA 87(1):83-87.
46. Fujisawa A, et al. (2007) Disease-associated mutations in CIAS1 induce cathepsin B-dependent rapid cell death of human THP-1 monocytic cells. Blood 109(7):2903-2911.
47. Perregaux D & Gabel C A (1994) Interleukin-1 beta maturation and release in response to ATP and nigericin. Evidence that potassium depletion mediated by these agents is a necessary and common feature of their activity. J Biol Chem 269(21):15195-15203.
48. Petrilli V, et al. (2007) Activation of the NALP3 inflammasome is triggered by low intracellular potassium concentration. Cell Death Differ 14(9):1583-1589.
49. Griffiths R J, Stam E J, Downs J T, & Otterness I G (1995) ATP induces the release of IL-1 from LPS-primed cells in vivo. J Immunol 154(6):2821-2828.
50. Shimada K, et al. (2012) Oxidized mitochondrial DNA activates the NLRP3 inflammasome during apoptosis. Immunity 36(3):401-414.
51. De Duve C & Wattiaux R (1966) Functions of lysosomes. Annu Rev Physiol 28:435-492.
52. Schroder B A, Wrocklage C, Hasilik A, & Saftig P (2010) The proteome of lysosomes. Proteomics 10(22):4053-4076.
53. Cuervo A M & Dice J F (1998) Lysosomes, a meeting point of proteins, chaperones, and proteases. Journal of molecular medicine 76(1):6-12.
54. Averette K M, et al. (2009) Anthrax lethal toxin induced lysosomal membrane permeabilization and cytosolic cathepsin release is Nlrp1b/Nalp1b-dependent. Plos One 4(11):e7913.
55. Droga-Mazovec G, et al. (2008) Cysteine cathepsins trigger caspase-dependent cell death through cleavage of bid and antiapoptotic Bcl-2 homologues. J Biol Chem 283(27):19140-19150.
56. Stoka V, et al. (2001) Lysosomal protease pathways to apoptosis. Cleavage of bid, not pro-caspases, is the most likely route. J Biol Chem 276(5):3149-3157.
57. Spreafico R, Ricciardi-Castagnoli P, & Mortellaro A (2010) The controversial relationship between NLRP3, alum, danger signals and the next-generation adjuvants. Eur J Immunol 40(3):638-642.
58. Harris J, Sharp F A, & Lavelle E C (2010) The role of inflammasomes in the immunostimulatory effects of particulate vaccine adjuvants. Eur J Immunol 40(3):634-638.
59. Kool M, et al. (2011) An unexpected role for uric acid as an inducer of T helper 2 cell immunity to inhaled antigens and inflammatory mediator of allergic asthma. Immunity 34(4):527-540.
60. Marichal T, et al. (2011) DNA released from dying host cells mediates aluminum adjuvant activity. Nature medicine 17(8):996-1002.
61. Kuroda E, et al. (2011) Silica Crystals and Aluminum Salts Regulate the Production of Prostaglandin in Macrophages via NALP3 Inflammasome-Independent Mechanisms. Immunity 34(4):514-526.
62. Seubert A, et al. (2011) Adjuvanticity of the oil-in-water emulsion MF59 is independent of Nlrp3 inflammasome but requires the adaptor protein MyD88. Proc Natl Acad Sci USA 108(27):11169-11174.
63. Kool M, et al. (2008) Alum adjuvant boosts adaptive immunity by inducing uric acid and activating inflammatory dendritic cells. J Exp Med 205(4):869-882.
64. Rock K L, Latz E, Ontiveros F, & Kono H (2010) The sterile inflammatory response. Annu Rev Immunol 28:321-342.
65. Kono H & Rock K L (2008) How dying cells alert the immune system to danger. Nature reviews. Immunology 8(4):279-289.
66. Brewer J M, et al. (1999) Aluminium Hydroxide Adjuvant Initiates Strong Antigen-Specific Th2 Responses in the Absence of IL-4- or IL-13-Mediated Signaling. The Journal of Immunology 163(12):6448-6454.
67. Cribbs D H, et al. (2003) Adjuvant-dependent modulation of Th1 and Th2 responses to immunization with beta-amyloid. International immunology 15(4):505-514.
68. Guy B (2007) The perfect mix: recent progress in adjuvant research. Nat Rev Microbiol 5(7):505-517.
69. Nakanishi K, Yoshimoto T, Tsutsui H, & Okamura H (2001) INTERLEUKIN-18 REGULATES BOTH TH1 AND TH2 RESPONSES. Annu Rev Immunol 19(1):423-474.
70. Chizzolini C, Chicheportiche R, Burger D, & Dayer J-M (1997) Human Th1 cells preferentially induce interleukin (m)-1β while Th2 cells induce IL-1 receptor antagonist production upon cell/cell contact with monocytes. Eur J Immunol 27(1):171-177.
71. Gavin A L, et al. (2006) Adjuvant-enhanced antibody responses in the absence of toll-like receptor signaling. Science 314(5807):1936-1938.
72. Wilson H L, Francis S E, Dower S K, & Crossman D C (2004) Secretion of intracellular IL-1 receptor antagonist (type 1) is dependent on P2X7 receptor activation. J Immunol 173(2):1202-1208.
73. Kawai T, Adachi O, Ogawa T, Takeda K, & Akira S (1999) Unresponsiveness of MyD88-deficient mice to endotoxin. Immunity 11(1):115-122.
74. Adachi O, et al. (1998) Targeted Disruption of the MyD88 Gene Results in Loss of IL-1- and IL-18-Mediated Function. Immunity 9(1):143-150.

1. A method of identifying an agent, or combination of agents, as a candidate immunological adjuvant comprising contacting a cell comprising a Nod-like receptor (Nlrp3) with the agent, combination of agents, quantifying the Nlrp3 response, comparing the Nlrp3 response to a predetermined level, and determining if the agent, or combination of agents, is a candidate immunological adjuvant, wherein the Nlrp3 response is pyroptosis and/or cytokine pro-I1-1β production and/or I1-1β release, and wherein the agent, or combination of agents, is a candidate immunological adjuvant if it effects a Nlrp3 response above a predetermined level of Nlrp3 response, and is not identified as a candidate immunological adjuvant if it effects a Nlrp3 response below the predetermined level of Nlrp3 response or if it does not effect a Nlrp3 response.

2. A method of identifying an agent, or combination of agents, as an immunological adjuvant comprising administering to a subject an agent, or combination of agents, identified as a candidate immunological adjuvant by the method of claim 1 and quantifying a subsequent Th1 response in the subject, and identifying the agent, or combination of agents, as an immunological adjuvant, wherein the agent, or combination of agents, is an immunological adjuvant if it effects a Th1 response in the subject above a predetermined level of Th1 response, and is not identified as an immunological adjuvant if it effects a Th1 response in the subject below the predetermined level of Th1 response or does not effect a Th1 response in the subject.

3. The method of claim 1, wherein the Nlrp3 response is pyroptosis.

4. The method of claim 1, wherein the Nlrp3 response is cytokine pro-I1-1β production or I1-1β release.

5. The method of claim 1, further comprising contacting a T-cell with the agent, or combination of agents, and determining T-cell proliferation, wherein an agent or combination of agents which effects T-cell proliferation is a candidate immunological adjuvant.

6. The method of claim 2, further comprising administering to the subject a vaccine or an antigen with the agent or with the combination of agents.

7. The method of claim 2, further comprising determining antibody production subsequent to the administering of agent, or combination of agents.

8. The method of claim 1, wherein the cell is a macrophage.

9. The method of claim 1, wherein the cell is a human macrophage.

10. The method of claim 1, wherein the combination of agents are used.

11. The method of claim 1, and wherein the combination of agents comprises at least one of a potassium efflux inducer, ATP, Bz-ATP, or nigericin.

12. A method of improving the efficacy of a vaccine comprising administering to a subject who is receiving, has received or will receive the vaccine, an amount of a secondary inducer of Nlrp3 effective to improve the efficacy of the vaccine.

13. The method of claim 12, wherein the secondary inducer of Nlrp3 is ATP, Bz-ATP, or nigericin.

14. The method of claim 12, wherein the secondary inducer is administered in a composition which also comprises the vaccine.

15. The method of claim 12, wherein the vaccine is a lipopolysaccharide (LPS) vaccine.

16. The method of claim 15, wherein the vaccine is a gram-negative bacteria lipopolysaccharide (LPS) vaccine.
